(19) United States
(12) Patent Application Publication
(10) Pub. No.: US 2020/0140496 A1
(43) Pub. Date: May 7, 2020

(54) METHOD FOR PREPARING A LIQUID EXTRACT OF PHYCOBILIPROTEINS, IN PARTICULAR PHYCOCYANIN, FROM CYANOBACTERIA OR MICROALGAE AND EXTRACT THUS OBTAINED

(71) Applicant: ALGOSOURCE, SAINT-NAZAIRE CEDEX (FR)

(72) Inventors: Olivier LEPINE, REZE (FR); Sébastien JUBEAU, GUERANDE (FR); Jérémy PRUVOST, SAINT BREVIN LES PINS (FR); Pascal JAOUEN, LE POULIGUEN (FR); Jean JENCK, SAINTE FOY LES LYON (FR)

(21) Appl. No.: 16/496,725

(22) PCT Filed: Mar. 22, 2018
(86) PCT No.: PCT/FR2018/050700

§ 371 Date: Dec. 18, 2019

(30) Foreign Application Priority Data
Mar. 24, 2017 (FR) 1752452

(51) Int. Cl. C07K 14/405; C07K 1/14; C07K 14/795

(52) U.S. Cl. CPC C07K 14/405; C07K 14/795; C07K 1/14

(57) ABSTRACT

A method for preparing a liquid extract rich in phycocyanin from cyanobacteria or microalgae containing phycocyanin in solution, comprising a step of carrying out the cellular lysis of an aqueous suspension of said fresh cyanobacteria or microalgae, a step of macerating the lysate obtained, for several hours in a solution of divalent cations, while releasing the water-soluble molecules in the extracellular space, and one or more steps of clarifying and concentrating the suspension in order to isolate the water-soluble molecules, among them phycocyanin. This method is performed at a pH between 5 and 8.5, at room temperature, without drying. Of

### FIELD OF THE INVENTION

The present invention relates to the field of processes for extraction of cyanobacteria or of microalgae with a view to obtaining biologically active compounds, more particularly for obtaining phycocyanin-rich extracts.

### PRIOR ART

Numerous methods have been developed over the past few years for extracting various types of molecules from cyanobacteria, one of the most well-known and widely used of which is spirulina.

This is because spirulina is rich in essential amino acids and in proteins (close to 50% by weight), in iron, and also in carbohydrates and lipids (gamma-linoleic acid) and in carotenoids (beta-carotene). It also contains numerous vitamins, in particular vitamins B1, B2, B6, B9, B12 and E, and also mineral trace elements (calcium, magnesium, zinc, potassium, etc.). The advantage of extracting these molecules is expanding for uses in the food, cosmetic and/or therapeutic fields.

Among proteins, phycobiliproteins are pigments which capture light energy. The main pigment of spirulina, which is phycocyanin, is a vivid blue. Phycobiliproteins also comprise allophycocyanin and phycoerythrin.

In the remainder of the text, the term “phycocyanin” is intended to mean “phycobiliproteins, in particular phycocyanin”.

There are numerous methods for extracting phycocyanin from spirulina. However, most of these processes use, as starting material, spirulina which has been dried (for example patent CN 101560254, or the publication by Robabeh et al, 2015, Journal of food processing and preservation, 39, pp 3080-3091, or by Silveira et al, 2007, Bioresource Technology, 98, pp 1629-1634) and/or optionally ground (FR 2 789 389), or else, if the extraction is carried out from fresh spirulina, the processes comprise a drying or freeze-drying step. However, it proves to be the case that, in the dry state, spirulina, and thus phycocyanin, are modified and their bioactive properties are degraded.

### OBJECTIVE OF THE INVENTION

A first objective of the invention is thus to provide a process for extracting phycocyanin, from fresh cyanobacteria, such as fresh spirulina, or from microalgae, which process comprises no step in which the product is in the dry state.

Some extraction processes use organic solvents to extract the phycocyanin, such as polyethylene glycol or glycerol (WO 2016/030643). These compounds are then, at least in trace amounts, in the final product. Since phycocyanin is intended for food and/or medical use, it is desirable not to use such methods which involve organic solvents. As a variant, the process must implement very expensive purification steps (for example chromatography).

Another objective of the present invention is thus to provide a process for preparing an extract of phycocyanin which does not involve organic solvents, or expensive purification steps.

A process for extracting phycocyanin using fresh cyanobacteria, but using large amounts of CaCl₂ calcium salts, of carbonates and of alumina sulfate and which result, after freeze-drying, in the obtaining of a blue powder, which can be preserved and stored, is known from document FR 2 453 199.

Another objective of the present invention is to provide a preparation process which limits the addition of salts, and makes it possible to obtain an extract of phycocyanin in liquid form, which extract is stable over time, without requiring freeze-drying or final drying.

Another objective of the process of the invention is to avoid any step which would bring about the precipitation of the phycocyanin in order to keep the latter in solution throughout the extraction process, the main objective being to obtain a phycocyanin in solution, which is non-denatured, so that it keeps its biological properties and its bioavailability.

### SUMMARY OF THE INVENTION

To this effect, the present invention provides a process for preparing a liquid extract of phycobiliproteins, and in particular of phycocyanin, in solution, from cyanobacteria or from microalgae containing phycocyanin, comprising the following successive steps:
i) a step of carrying out cell lysis, by means of a physical or mechanical method, of an aqueous suspension of said fresh cyanobacteria or microalgae,
ii) a step of macerating the lysate obtained in step i) in a solution of divalent cations, preferably alkaline-earth metal cations, with a view to releasing the water-soluble molecules in the extracellular space of the cyanobacteria or of the microalgae,
iii) one or more steps of clarifying and concentrating the suspension in order to isolate the water-soluble molecules, among them the phycocyanin, all of this process being carried out at a pH of between 5 and 8.5, without drying and at a temperature of less than or equal to 25° C. allowing the phycocyanin to retain its spatial structure, thus preserving its biological properties and giving it better bioavailability.

The extract obtained is thus an aqueous extract, without any trace of organic solvent, since no step of said process of the invention comprises introducing such molecules for the purpose of extracting the phycocyanin.

The process according to the invention comprises neither a precipitation step nor a drying step, which allows the phycocyanin to remain in solution, to not be denatured and to retain its spatial structure, thus preserving its biological properties and giving it better bioavailability, as presented below in the examples.

The term “physical or mechanical method” is intended to mean the fact that this destructuring or cell lysis step i) comprises the addition of no chemical or biological additive, as opposed in particular to the process of FR 2 929 957 which aims to obtain cyanobacteria highly loaded with metal (spirulina-metal) and the first step of which is a physiological induction by application of a stress and the suppression of oxygen by means of NaCl and of sodium sulfite, before placing the cyanobacteria in the presence of a divalent metal. This results in a partial denaturation of the phycocyanin, which goes against the process according to the present invention.

Preferably, the destructuring or cell lysis step comprises a freezing-thawing phase.

The freezing-thawing phase makes it possible in particular to weaken the cell walls and membranes, in order to subsequently facilitate the extraction of the intracellular metabolites. Advantageously, the freezing-thawing phase comprises the freezing and the preservation of the frozen cyanobacteria or microalgae at a temperature of less than −18° C., preferably less than −20° C., more preferably less than −24° C., for a period ranging from one day to one year, preferably from two weeks to six months; this freezing is followed by a step of slow thawing at a temperature of greater than 0° C., and preferably less than 5° C., for several hours.

The step of macerating the lysate obtained in step i) is preferentially carried out with stirring of said suspension thermostatted at a temperature of between 10° C. and 25° C. for several hours: advantageously for at least 3 hours, preferably for at least 4 hours, more preferably for at least 6 hours.

The clarification step is advantageously carried out by centrifugation, then recovery of the supernatant solution. The objective of this clarification is to reduce a large part of the particulate fraction present in the suspension obtained in step ii). These particles are essentially cell debris (walls, membranes) and metabolites which have low water-solubility or are water-insoluble (lipids, hydrophobic proteins, etc.). It has been noted that the presence of CaCl₂ greatly promotes the decanting of the particles by increasing their sedimentation rate during centrifugation.

Advantageously, the supernatant solution resulting from the centrifugation step is subjected to a microfiltration step, preferably a tangential microfiltration, by means of a membrane having a cut-off threshold of between 0.1 μm and 2 μm, preferably between 0.1 μm and 1.4 μm, more preferably between 0.2 μm and 1 μm, then recovery of the filtrate.

This microfiltration step makes it possible, firstly, to complete the clarification of the aqueous extract by removing all the particles not decanted during the centrifugation, and, on the other hand, to reduce the bacterial load of the product (sterilizing filtration).

The water-soluble fraction, comprising in particular proteins, including phycocyanin, phycobiliproteins, and sugars will pass through the membrane and be in the filtrate (permeate).

This filtrate of the microfiltration step is then advantageously subjected to a separation by ultrafiltration, preferably a tangential ultrafiltration, by means of a membrane with a cut-off threshold of between 1 and 50 kDa, preferably of between 5 and 25 kDa, making it possible to separate the phycocyanin from the small water-soluble molecules and to collect an aqueous solution enriched with phycocyanin.

Thus, this operation constitutes a step of purifying the phycocyanin, since the phycocyanin is retained on the membrane which allows the other smaller water-soluble molecules to pass through (in particular peptides, small sugars, salts). It also makes it possible to reduce the volume of the clarified solution of phycocyanin, by concentrating it. In concentrated form, the phycocyanin solutions are then more stable during cold storage.

Thus, the phycocyanin-enriched solution obtained after the ultrafiltration step makes it possible to contain a phycocyanin concentration of greater than or equal to 0.5 g/l, preferably greater than or equal to 1 g/l, more preferably greater than or equal to 2 g/l, more preferably greater than or equal to 10 g/l. The phycocyanin content in said phycocyanin solution is advantageously determined by measuring the optical density at one or more wavelengths of between 615 nm and 750 nm.

With regard to the aqueous solution of divalent cations of step ii), it advantageously contains between 10 mM and 100 mM, preferably between 10 mM and 60 mM, more preferably between 15 mM and 55 mM, of divalent cations. The addition of the aqueous solution of divalent cations allows better migration of the water-soluble molecules in the extracellular space. The divalent cations are preferably calcium ions, more particularly in the form of calcium chloride.

If necessary, the pH is adjusted to a value of between 5 and 7.5, preferably to a value of between 5.5 and 7.0.

The process for preparing a phycocyanin-rich liquid extract according to the present invention can be carried out starting from cyanobacteria chosen from the spirulina Arthrospira platensis, Aphanizomenon flos-aquae, or Phormidium molle, or any other phycocyanin-containing cyanobacterium.

The present invention also relates to a liquid extract of cyanobacteria or of microalgae, prepared by means of the process described above, comprising a content of phycocyanin in solution, which is non-denatured, of greater than or equal to 1 g/l, preferably greater than or equal to 2 g/l, more preferably greater than or equal to 10 g/l. This aqueous extract is clear and stable over time, not exhibiting any sedimentation for several months of preservation (at ambient temperature). It can be used in the form of a beverage.

### BRIEF DESCRIPTION OF THE DRAWINGS

The invention will be clearly understood from reading the following description of examples of implementation, with reference to the appended figures wherein:

FIG. 1 is a schematic diagram of the main steps of the process for preparing a phycocyanin-rich aqueous extract according to the present invention;

FIG. 2 shows the phycocyanin extraction yield as a function of the maceration time for a CaCl₂ content of 10 mM;

FIG. 3 shows the results of measurement of the total antioxidant status (TAS) on three groups of hamsters after 4 weeks and 11 weeks subjected to various diets, one of which included the extract according to the invention;

FIG. 4 shows the results of measurement of superoxide dismutase (SOD) on three groups of hamsters after 4 weeks and 11 weeks of diet;

FIG. 5 shows the results of measurement of glutathione peroxidase (GPx) on three groups of hamsters after 11 weeks of diet; and

FIG. 6 shows the result of measurement of the plasma levels of malondialdehyde (MDA) on three groups of hamsters after 11 weeks of diet.

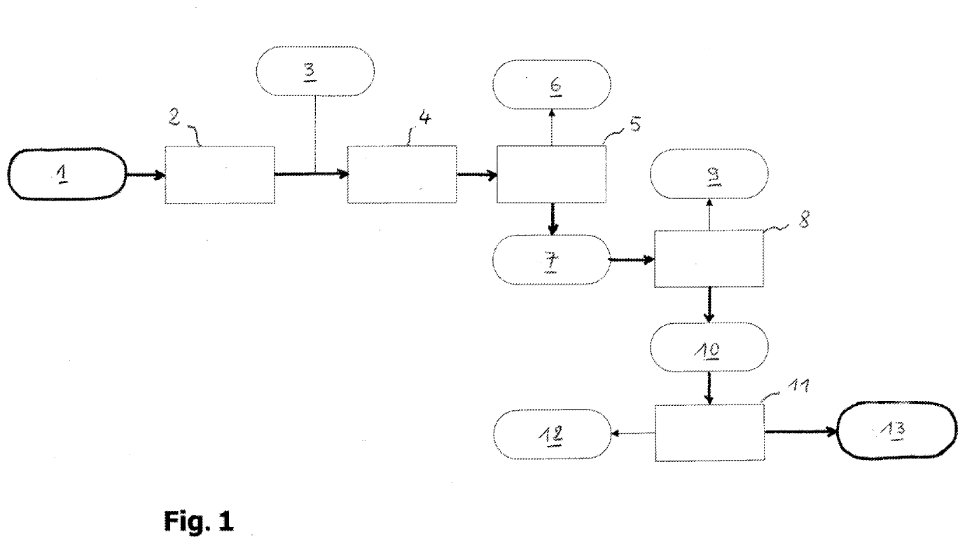

### EXAMPLES

### Example 1

In this nonlimiting example illustrating the invention, the starting fresh cyanobacteria 1 are from the family of the spirulina Arthrospira platensis cultured in bioreactors in a briny aqueous medium. These microalgae are collected and drained on a sieve with a mesh of 50 μm. The solids content of this biomass is 20% by weight.

The biomass is first of all subjected to a slow freezing step 2 (less than −1° C./min down to a temperature of −20° C.) and preserved at this temperature for 3 to 12 months, advantageously for reasons of seasonal production of spirulina.

The preservation of the biomass in frozen form has the double advantage of ensuring the non-denaturation of compounds of interest contained in the cells, but also of causing destructuring of the biomass. This destructuring results from various phenomena involved during freezing, namely the formation of intracellular and extracellular ice, osmotic dehydration and ice crystal rearrangement leading to high mechanical stresses on the cells. The latter phenomenon is quite slow and requires a significant period of freezing time in order to ensure that the main barriers to extraction, namely the various membranes and cell wall, are indeed destructured. After thawing, good penetration of the extraction solvent and, by the same token, good extraction yields, are thus ensured.

After this period of storage at very low temperature, the biomass is thawed to a temperature of greater than 0° C. by keeping it in a chamber at approximately +3° C. This low, but positive, temperature allows slow thawing which promotes cell permeabilization.

Next, the objective of the aqueous extraction is to release the phycocyanin from the cell envelope in order to find it in solution in the extracellular aqueous phase. For this purpose, the thawed biomass is first of all suspended in an aqueous solution (solid/liquid weight proportion: 15/85). This aqueous solution 3 comprises water microfiltered at 0.2 μm and calcium chloride CaCl₂ at 10 mM. The pH is adjusted if necessary to 7. The solution thermostatted at 20° C. is subjected to a maceration 4 with stirring for 7 hours in the dark.

The presence both of a large amount of water relative to the biomass and of calcium chloride allows migration of most of the water-soluble molecules in the extracellular space. More than 95%, or even 99%, of the phycocyanin present in the initial biomass is then found in this extracted fraction (cf. example 2 below).

This suspension is then subjected to a centrifugation step 5 (15 000 g, for 10 minutes at 20° C.) which makes it possible to discard a large portion of the particulate fraction present (cell debris, and metabolites which have low water-solubility or are water-insoluble). It has been noted that the presence of calcium chloride promotes the settling out of these particles. The algal residue 6 is discarded and only the blue-colored supernatant solution 7 is then preserved.

This solution 7 is then subjected to a further clarification step. For this, a tangential microfiltration operation 8 is carried out. The cut-off threshold is 0.2 pm (it is thus a sterilizing filtration). The water-soluble fraction (phycocyanin and sugars in particular) passes through this membrane and is found in the permeate 10. The retentate 9 is discharged.

The ultrafiltration step 11 which follows has the objective of concentrating the volume of this phycocyanin solution and of removing the contaminating small molecules. It is a tangential ultrafiltration with a cut-off threshold of 10 kDa. This cut-off threshold retains the phycocyanin while at the same time allowing the other, smaller, water-soluble molecules (peptides, small sugars, salts) to pass through in the permeate 12. This step thus also constitutes a phycocyanin purification step. A clear, concentrated and purified phycocyanin solution 3 is thus obtained.

The phycocyanin content (of several grams per liter which can reach up to 50 g/l) is determined by measuring the optical density at 615 nm, 652 nm and 750 nanometers (see formula I of example 2). This phycocyanin solution is stable for several months without the addition of stabilizer or preservative, and can be packaged in sterile vials.

### Example 2

### Phycocyanin Yield

Frozen spirulina paste containing approximately 20% solids is placed in water at ambient temperature and at a CaCl₂ concentration of 10 mM (1.1 g/l) according to the ratio 15/85 (m/m). The mixture is then placed in the dark with stirring.

A first fraction of this suspension obtained is centrifuged (10 min, T_{ambient}, 13000×g) in order to monitor the extraction of the phycocyanin (PC). The supernatant aqueous extract thus obtained is then analyzed on a spectrophotometer by measuring the absorbance at various wavelengths: respectively 615 nm, 652 nm and 750 nm. The phycocyanin concentration is calculated using the following equation (I) (according to Bennett and Bogorad, J. Cell. Biol., 419-435, 1973):

$${[ PC ]}_{g / l} = \frac{( {Abs}_{615 n m} - {Abs}_{750 n m} ) - 0.474 ( {Abs}_{652} - {Abs}_{750 n m} )}{5.34} \qquad ( I )$$

In order to calculate the extraction yields, the total amount of phycocyanin must be determined. To do this, a second fraction of the same suspension of spirulina was treated in a cell destroyer with high dynamic pressures, which makes it possible to obtain total lysis of the cells and release of all the water-soluble molecules contained in the cells. The ground material thus obtained is subsequently clarified and then analyzed by spectrophotometry like the previous aqueous extracts.

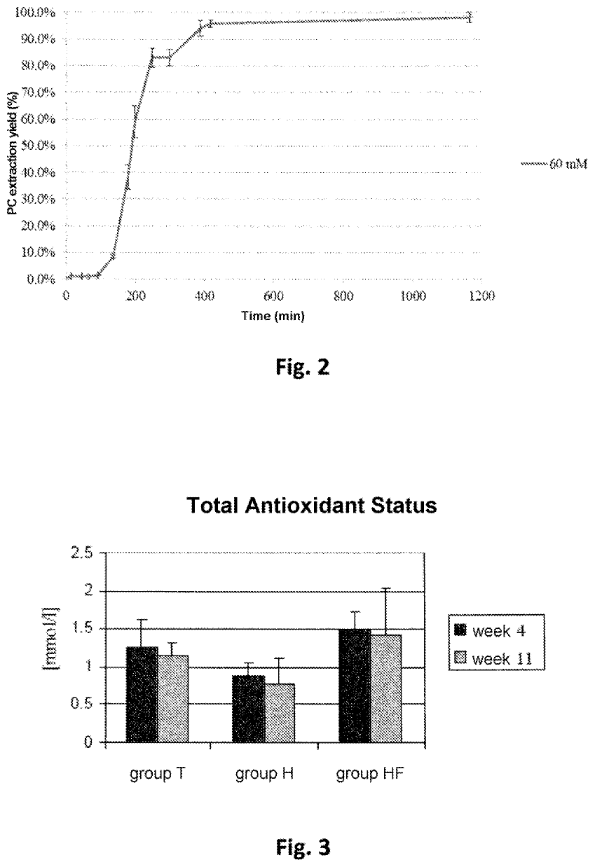

The results showing the phycocyanin extraction yield as a function of the extraction time are presented in appended FIG. 2.

After 400 minutes of extraction according to the protocol detailed above, it is noted in particular that more than 95% of the total phycocyanin has been extracted according to the process of the present invention.

### Example 3

### In Vitro Properties

In this example, the effect of the phycocyanin degradation during a drying step was measured by measuring the SOD (superoxide dismutase) activity by means of the Bioxytech SOD-525 kit sold by Cayman Chemical (USA).

Two identical samples of phycocyanin extracted according to the process described in example 1 above were compared:

A—a 5 ml vial of phycocyanin in solution containing 9.50 mg of phycocyanin. The equivalent SOD activity measured is 181 U/ml, i.e. for 10 mg of phycocyanin, an activity of 953 U SOD equivalent.

B—the phycocyanin extract was dehydrated at low temperature, i.e. below 30° C., until a powder was obtained. This powder contained 350 mg of phycocyanin per gram. The measurement of the equivalent SOD activity equivalent indicated a value of 633 U/g, i.e. related back to 10 mg of phycocyanin, a value of 18 U SOD equivalent.

The equivalent SOD activity of the phycocyanin having undergone no drying step is thus 953/18=53 times higher than the same dry product.

### Example 4

### In Vivo Properties

An aqueous extract of spirulina was prepared according to the process as described in example 1. This extract contains 1 g/l of phycocyanin.

The antioxidant activity of this spirulina extract was demonstrated by a study carried out for 11 weeks on three groups of 6 hamsters having followed various diets:
Groupe T: subjected to a standard diet
Groupe H: subjected to a high fat diet
Groupe HF: subjected to a high fat diet with a liquid spirulina supplementation (phycocyanin at 1 g/l: the spirulina extract is provided in the drinking water of the hamsters at a dose of 1 ml/day).

Since the high-fat diet is known to induce oxidative stress, the result of this stress is determined by various measurements carried out in week no. 4 and/or in week no. 11:

Measurement of the Total Antioxidant Status (TAS):

The total antioxidant status measured with the TAS-NX 2332 kit (Randox Laboratories Ltd) reflects the activity potential of the antioxidant system making it possible to protect the tissues against the effects of free radicals. Results presented in FIG. 3 show that the phycocyanin-rich extract supplementation results in a better antioxidant status, which makes it possible to increase the capacity of the cell to defend itself against damage caused by oxidative stress.

Measurement of Superoxide Dismutase (SOD):

Superoxide dismutase is the first enzyme of the free-radical reduction chain. The SOD is the enzyme required for maintaining life in the presence of oxygen; it makes it possible to eliminate the active oxygen species that are toxic to cells. The SOD is measured on the blood by means of the Ransod—SD 125 kit (Randox Laboratories Ltd).

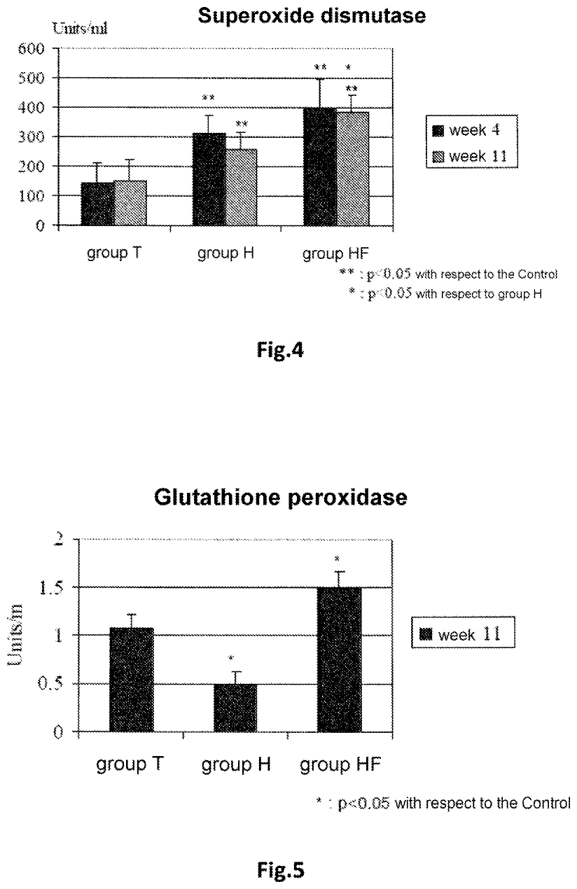

The amount of SOD present in the blood is increased by 30% to 50% in the hamsters subjected to the diet with spirulina extract (see FIG. 4). The spirulina extract increases the amount of SOD available in the blood by stimulating its production.
Measurement of Glutathione Peroxidase (GPx):

Glutathione peroxidase has the property of reducing oxidized free radicals by oxidizing reduced glutathione (GSH) to glutathione. It is measured with the Ransel—RS 505 kit (Randox Laboratories Ltd).

At week 11, a significant increase in the GPx activity is noted (see FIG. 5) by virtue of the spirulina extract according to the invention which thus allows effective control against the free radicals.

Measurement of Lipid Peroxidation:

Malondialdehyde (MDA) is naturally present in the tissues, it is one of the products of the fatty acids oxidation; a high level is a marker for oxidative stress. It is measured on the plasma with the MDA kit, ref. 1203.001 (Sobioda).

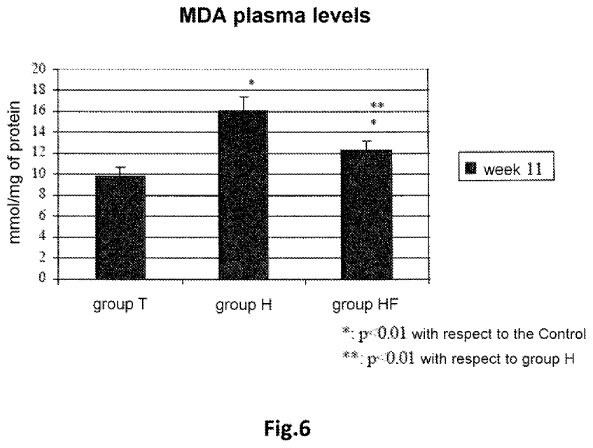

The results presented in FIG. 6 show that the spirulina extract supplementation allows a decrease in the level of MDA and thus an inhibition of the lipid peroxidation, allowing a reduction in the oxidative stress.

All of the results above thus show that the spirulina extract prepared according to the process of the present invention has good bioavailability and an antioxidant activity demonstrated in vivo.

1. A process for preparing a liquid extract of phycobiliproteins, and in particular of phycocyanin, in solution, from cyanobacteria or from microalgae containing phycocyanin, comprising the following successive steps:
i) a step of carrying out cell lysis, by a physical or mechanical method, of an aqueous suspension of fresh cyanobacteria or microalgae,
ii) a step of macerating the lysate obtained in step i) in a solution of divalent cations, preferably alkaline-earth metal cations, with a view to releasing the water-soluble molecules in the extracellular space of the cyanobacteria or of the microalgae,
iii) one or more steps of clarifying and concentrating the suspension in order to isolate the water-soluble molecules, among them the phycocyanin, all of this process being carried out at a pH of between 5 and 8.5, without drying and at a temperature of less than or equal to 25° C. allowing the phycocyanin in solution to retain its spatial structure, thus preserving its biological properties and giving it better bioavailability.

2. The process as claimed in claim 1, characterized in that the destructuring or cell lysis step comprises a freezing-thawing phase.

3. The process as claimed in claim 2, characterized in that the freezing-thawing phase comprises the freezing and the preservation of the frozen cyanobacteria or microalgae at a temperature of less than −18° C., preferably less than −20° C., more preferably less than −24° C., for a period ranging from one day to one year, preferably from two weeks to six months, followed by a step of slow thawing at a temperature of greater than 0° C., and preferably less than 5° C., for several hours.

4. The process as claimed in any one of the preceding claims, characterized in that the step of macerating the lysate obtained in step i) is carried out with stirring of said suspension thermostatted at a temperature of between 10° C. and 25° C. for several hours.

5. The process as claimed in any one of the preceding claims, characterized in that the clarification step is carried out by centrifugation, then recovery of the supernatant solution.

6. The process as claimed in claim 5, characterized in that the supernatant solution resulting from the centrifugation step is subjected to a microfiltration step, preferably a tangential microfiltration, by means of a membrane having a cut-off threshold of between 0.1 μm and 2 μm, preferably between 0.1 μm and 1.4 μm, more preferably of between 0.2 μm and 1 μm, then recovery of the filtrate.

7. The process as claimed in claim 6, characterized in that the filtrate of the microfiltration step is subjected to a separation by ultrafiltration, preferably a tangential filtration, by means of a membrane with a cut-off threshold of between 1 and 50 kDa, preferably of between 5 and 25 kDa, making it possible to separate the phycocyanin from the small water-soluble molecules and to collect an aqueous solution enriched with phycocyanin.

8. The process as claimed in claim 7, characterized in that the phycocyanin-enriched solution obtained after the ultrafiltration step contains a phycocyanin concentration of greater than or equal to 0.5 g/l, preferably greater than or equal to 2 g/l, more preferably greater than or equal to 10 g/l, the phycocyanin content in said phycocyanin solution being determined by measuring the optical density at one or more wavelengths of between 615 and 750 nm.

9. The process as claimed in any one of the preceding claims, characterized in that the aqueous solution of divalent cations of step ii) contains between 10 mM and 100 mM, preferably between 10 and 60 mM, more preferably between 15 mM and 55 mM, of divalent cations.

10. The process as claimed in any one of the preceding claims, characterized in that the pH is adjusted to a value of between 5 and 7.5, preferably to a value of between 5.5 and 7.

11. The process as claimed in any one of the preceding claims, characterized in that the divalent cations are calcium ions.

12. The process as claimed in any one of the preceding claims, characterized in that the cyanobacteria are chosen from the spirulina Arthrospira platensis, Aphanizomenon flos-aquae, or Phormidium molle.

13. A liquid extract of cyanobacteria or of microalgae, prepared by means of the process as claimed in any one of the preceding claims, comprising a content of phycocyanin in solution, which is non-denatured, of greater than 1 g/l, preferably greater than or equal to 2 g/l, more preferably greater than or equal to 10 g/l.
